(19) United States
(12) Patent Application Publication
(10) Pub. No.: US 2026/0102203 A1
(43) Pub. Date: Apr. 16, 2026

(54) ROTATABLE MEDICAL DEVICES AND RELATED METHODS

(71) Applicant: Boston Scientific Scimed, Inc., Maple Grove, MN (US)

(72) Inventors: Katherin NARVAEZ NUÑEZ, Cartago (CR); Juan Pablo ORTIZ GARCIA, Heredia (CR); Jimena CESPEDES BERROCAL, Alajuela (CR); Pablo Arnoldo MONTERO RODRÍGUEZ, San Jose (CR); Cristopher OVIEDO MIRANDA, Alajuela (CR); Cristian ARAYA CAMACHO, Alajuela (CR); Sebastian MURILLO MURILLO, Cartago (CR)

(73) Assignee: Boston Scientific Scimed, Inc., Maple Grove, MN (US)

(21) Appl. No.: 19/351,597

(22) Filed: Oct. 7, 2025

Related U.S. Application Data
(60) Provisional application No. 63/707,383, filed on Oct. 15, 2024.
(60) Provisional application No. 63/717,361, filed on Nov. 7, 2024.

(51) Int. Cl. A61B 18/14; A61B 18/00; A61B 90/00

(52) U.S. Cl. CPC A61B 18/1445; A61B 90/03; A61B 2018/00083; A61B 2018/00172; A61B 2018/00202; A61B 2018/0091; A61B 2018/00982; A61B 2090/034

(57) ABSTRACT

A medical device includes a shaft and a wire that extends radially within the shaft. The medical device also includes an actuator and an end effector assembly at a distal end of the shaft. The wire is coupled to the actuator and the end effector assembly, and the wire transmits a force to the end effector assembly. A connection between the end effector assembly and the shaft permits axial rotation of the end effector assembly. The connection includes a flange protruding radially outward relative to a position of the wire toward a gap and a clevis. The wire is coupled to the clevis.

### CROSS-REFERENCE TO RELATED APPLICATIONS

This application claims the benefit of priority to U.S. Provisional Application No. 63/707,383, filed on Oct. 15, 2024, and U.S. Provisional Application No. 63/717,361, filed on Nov. 7, 2024, each of which is incorporated by reference herein in its entirety.

### TECHNICAL FIELD

Various embodiments of this disclosure relate generally to rotatable medical devices, for example, medical devices having a rotatable end effector, and related methods.

### BACKGROUND

In some procedures, a physician may require precise manipulation of an end effector. For example, during surgeries, a physician may use accessory devices, such as forceps, baskets, nets, and other devices, to access and treat a target site within a patient. The physician or a technician typically introduces an accessory device into a working channel of a scope, such as an endoscope, to reach the target site. Once the accessory device reaches the target site, the physician may proceed with the procedure. However, some devices may not rotate the end effector independently from the handle with precision. In some procedures, limited manipulation of the end effector may be disadvantageous, leading to increased procedure times, difficulties accessing the treatment site, or delivering effective treatment.

### SUMMARY

Aspects of the disclosure relate to, among other things, devices, and methods to enable actuation and rotation of a distal end effector to enable treatment at a target tissue site within a subject (e.g., patient). Each of the aspects disclosed herein may include one or more of the features described in connection with any of the other disclosed aspects.

In one example, a medical device may comprise a shaft, a wire that may extend radially within the shaft, an actuator, an end effector assembly at a distal end of the shaft, and a connection. The wire may be coupled to the actuator and the end effector assembly, and the wire may transmit a force to the end effector assembly. The medical device may comprise a connection between the end effector assembly and the shaft, which may permit axial rotation of the end effector assembly. The connection may include a flange protruding radially outward relative to a position of the wire toward a gap and a clevis. The wire may be coupled to the clevis.

Any examples described herein may have any of these features alone or in combination. The shaft may be coupled to the bushing, and the bushing may be distal to the shaft. The gap may be defined between the flange and an inner surface of the bushing. The flange may be axially translatable relative to the shaft to engage a distally-facing inner surface of the bushing. The clevis and the wire may rotate relative to the bushing and the shaft. The medical device may further comprise a cannula, and the flange may protrude from an inner surface of the cannula. The bushing may include a cavity, and the cannula may be coupled to a radially outward facing surface of the cavity. The cannula may be axially translatable relative to the shaft to engage a distally-facing inner surface of the cavity. The gap may be defined between the flange of the cannula and the inner surface of the cavity of the bushing. The cannula may be fixedly coupled to the clevis, and a space may separate the clevis and the bushing. The end effector assembly may rotate when moved by the actuator, and the clevis, the wire, and the cannula may rotate relative to the shaft during rotation. The shaft may include a coil, and at least an inner surface of the coil may include a non-conductive coating. The bushing may be fixed to an outer surface of a distal portion of the coil. The distal end of the clevis may include the flange, and the bushing may include the gap and the flange. The flange may extend radially outward from the clevis and within the gap, and the distal portion of the coil may be adjacent a space that separates the coil from the clevis.

The medical device may further comprise a handle body. The actuator may be rotatably coupled to the handle body. The shaft may be fixedly coupled to the actuator and extend to the end effector assembly for actuation of the end effector assembly. The handle body may include an extension having a radial protrusion, and the actuator may include an opening that receives the radial protrusion. A crimp may be positioned on the shaft and positioned within a recess in the actuator. The actuator, the shaft, the wire, and the clevis may rotate relative to the handle body and the extension of the handle body.

In another example, a medical device may comprise a handle body. The handle body may include a rotation actuator and a longitudinal actuator. The rotation actuator may be rotatably coupled to the handle body, and the longitudinal actuator may be movably coupled to the handle body. The medical device may also include a wire, a shaft, and an end effector assembly. The proximal portion of the wire may be fixedly coupled to the longitudinal actuator, and another portion of the wire may be fixedly coupled to the rotation actuator. The wire may extend radially within the shaft and the wire may extend to the end effector assembly. The end effector assembly may comprise a bushing, a cannula, and a clevis. The bushing may be coupled to a distal end of the shaft, and the bushing may include a cavity. The cannula may be positioned within the cavity of the bushing, and the cannula may include a flange extending radially outward toward a gap in the cavity. Movement of the longitudinal actuator may move the wire and the clevis longitudinally relative to the handle body, and rotation of the rotation actuator may rotate the wire, the cannula, and the clevis relative to the handle body, the shaft, and the bushing.

Any of the examples disclosed herein may include any of the following features in any combination. A proximal portion of the bushing may include an increased thickness. The bushing may taper in a distal direction, and an inner wall of the bushing may form a stop surface configured to inhibit distal movement of the cannula. The gap may be positioned between the flange and an inner surface of the wall of the cavity. The cannula may be fixedly coupled to a proximal portion of the clevis. Rotation of the rotation actuator may rotate the wire and the clevis, and rotation of the wire may rotate the flange within the gap.

In another example, a medical device may comprise a handle body, which may include a longitudinal actuator. The medical device may include a wire, and a proximal portion of the wire may be fixedly coupled to the longitudinal actuator. The medical device may include a shaft, and the wire may extend radially within the shaft. In addition, the medical device may include an end effector assembly. The end effector assembly may include a clevis and a bushing. The wire may be fixedly coupled to the clevis, and the bushing may be coupled to a distal end of the shaft. The clevis may be coupled to a distal end of the bushing. The rotation of the handle body may rotate the shaft to rotate the bushing, and rotation of the handle body may rotate the wire to rotate the clevis.

According to some aspects, rotation of the handle body may rotate the shaft, the bushing, the wire, and the clevis in a same direction.

It may be understood that both the foregoing general description and the following detailed description are exemplary and explanatory only and are not restrictive of the disclosure, as claimed.

### BRIEF DESCRIPTION OF THE DRAWINGS

The accompanying drawings are incorporated herein and constitute a part of this specification, illustrate exemplary aspects of the disclosure, and together with the description, explain the principles of the disclosure.

FIG. 1 is a side view of a medical device, according to aspects of this disclosure;

FIG. 2 is an enlarged, cross-sectional view of internal portions of the handle assembly of FIG. 1, according to aspects of this disclosure;

FIG. 3A is a schematic illustration of the medical device of FIG. 1, according to aspects of this disclosure;

FIG. 3B is a schematic illustration of a transversal section of an actuator of the medical device of FIG. 1;

FIG. 3C is a schematic illustration of an exemplary distal portion of the medical device of FIG. 1, according to aspects of this disclosure;

FIG. 4 is a schematic illustration of an exemplary distal portion of a medical device, according to aspects of this disclosure;

FIG. 5A is a side cross-sectional view of an exemplary actuator, according to aspects of this disclosure;

FIG. 5B is a side cross-sectional view of a portion of an exemplary handle of a medical device, according to aspects of this disclosure; and FIG. 5C is a side cross-sectional view of an exemplary distal portion of a medical device, according to aspects of this disclosure;

FIG. 6 is a partially transparent view of a proximal portion of an exemplary medical device, according to aspects of this disclosure; and

FIG. 7 is a partially transparent view of a distal portion of the medical device of FIG. 6, according to aspects of this disclosure.

### DETAILED DESCRIPTION

Aspects of the disclosure include devices and methods to enable actuation and rotation of a distal end effector of a medical device, which may help to enable treatment at a target tissue site within a subject (e.g., patient). The ability to manipulate (including rotate) the end effector in a precise manner enables a user to grasp or otherwise manipulate a target more efficiently, thereby reducing procedure time, more precise grasping/manipulation of tissue, and a decreased volume of blood loss. Accordingly, the devices and methods disclosed herein may help result in better patient outcomes.

The medical device may be introduced into the body without a delivery device or via a delivery device. The delivery device may be a catheter, a scope (endoscope, bronchoscope, colonoscope, duodenoscope, etc.), a tube, a sheath, or other like device, inserted into a body cavity or lumen, for example the GI tract, via a natural orifice. The orifice can be, for example, the nose, mouth, or anus, and the placement can be in any portion of the GI tract, including the esophagus, stomach, duodenum, large intestine, or small intestine. Delivery and placement also can be in other body lumens or organs reachable via the GI tract, any natural opening, any other body tract, or any bodily incision.

Furthermore, wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the subject. As used herein, the terms “comprises,” “comprising,” or any other variation thereof, are intended to cover a non-exclusive inclusion, such that a process, method, article, or apparatus that comprises a list of elements does not necessarily include only those elements but may include other elements not expressly listed or inherent to such process, method, article, or apparatus. The term “exemplary” is used in the sense of “example,” rather than “ideal.” As used herein, the terms “about,” “substantially,” and “approximately,” indicate a range of values within +/−10% of a stated value. As used herein, the phrase “based on” is understood to be equivalent to the phrase “based at least on,” unless indicated otherwise. The term “or” is used disjunctively, such that “at least one of A or B” includes, (A), (B), (A and A), (A and B), (B and B), etc.

Examples of the disclosure may relate to devices and methods for performing various medical procedures and/or treating portions of the large intestine (colon), small intestine, cecum, esophagus, any other portion of the gastrointestinal tract, and/or any other suitable patient anatomy (collectively referred to herein as a “target treatment site”). Various examples described herein include single-use or disposable medical devices. Any structures of the medical devices described herein can be made of biocompatible materials, including biocompatible polymers, rubbers, plastics, and the like.

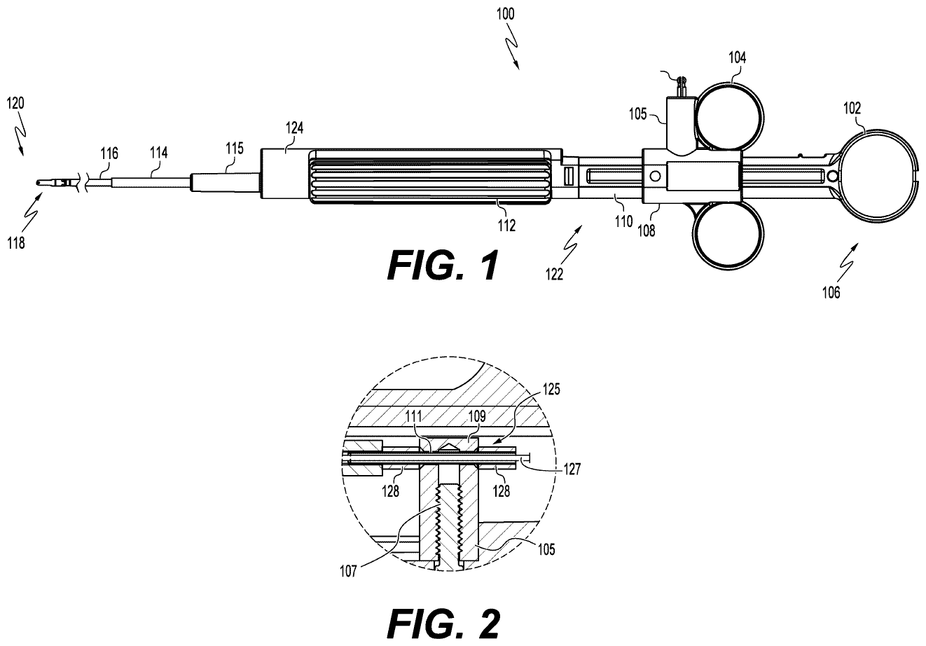

Reference will now be made in detail to aspects of the disclosure, examples of which are illustrated in the accompanying drawings. Embodiments of this disclosure relate to aspects of a distal portion of a medical device and actuators of a medical device configured to provide rotation to an end effector assembly. The end effector of the medical device may rotate about its axis and relative to aspects of the medical device. FIG. 1 and FIG. 2 depict aspects of the medical device and its handle.

FIG. 1 illustrates a medical device 100 including a proximal portion 106 and a distal portion 120. Medical device 100 comprises a handle assembly 122 and a shaft 116. In some aspects, shaft 116 includes an end effector assembly 118, for example, at distal portion 120. Handle assembly 122 includes an electrical connection or insert 107, which may be electrically connected to a lumen and/or one or more channels of shaft 116. Additionally, handle assembly 122 may include an actuator 108, which may be a longitudinal actuator used to actuate or control one or more aspects of end effector assembly 118, as described in detail below. Electrical insert 107, including a protrusion 105, which may be cylindrical. Electrical insert 107 may be positioned on actuator 108 or alternatively on another portion of handle assembly 122, for example, a handle body 110, also described in the subsequent sections. As discussed below, handle assembly 122 also includes a rotation actuator 112 that may control the rotation of one or more components or portions of distal portion 120 of medical device 100.

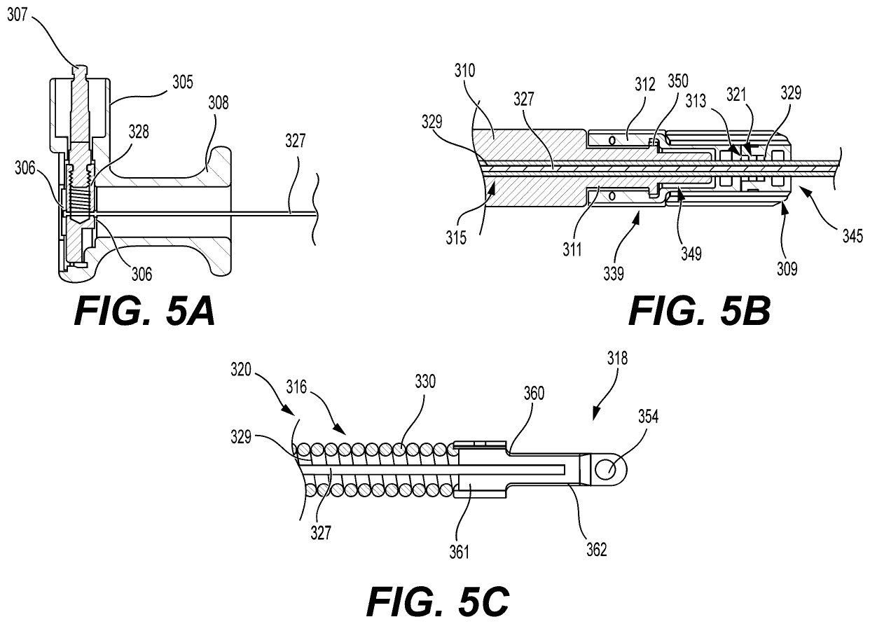

Actuator 108 may include one or more finger rings or slot(s) 104. For example, two finger slots 104 may extend outwardly (e.g., radially outward) from a proximal portion of actuator 108. Finger slot(s) 104 may be proximal to or otherwise adjacent to port 107. The one or more finger slot(s) 104 may be any suitable shape and size to enable a user to insert a finger through or otherwise position a finger on or within at least a portion of the slot 104. For example, the one or more finger slot(s) 104 may be circular (as shown), triangular, rectangular, cylindrical or any shape commonly known in the art. However, the disclosure is not so limited and actuator 108 may include indentations or any other suitable configuration known in the art (FIG. 5A).

Additionally, handle assembly 122 may include handle body 110, which may be integrally formed within handle assembly 122. Handle body 110 may include a thumb or proximal ring 102 located at or near a proximal end of handle assembly 122. Proximal ring 102 may be shaped similar to or from the one or more finger slots 104 on actuator 108. Handle body 110 may include a slot, opening, or track 113, for example, extending longitudinally through a portion of handle body 110. Actuator 108 may be moveably coupled to handle body 110 via track 113. For example, a portion of actuator 108 may extend through track 113 (e.g., laterally), and actuator 108 may be movable along or within track 113. The movement of actuator 108 relative to handle body 110 may actuate or control an end effector assembly 118, as discussed below.

For example, actuator 108 may translate within track 113 in a first direction, for example, towards a distal end of handle assembly 122. Additionally, actuator 108 may translate within track 113 in a second direction, for example, towards a proximal end of handle assembly 122. The movement of actuator 108 in these directions results in the actuation or de-actuation of end effector assembly 118 (e.g., opening and closing). As discussed below, a wire 127 may be coupled to actuator 108, such that movement of actuator 108 in the first direction or in the second direction imparts movement or other actuation to end effector assembly 118.

Furthermore, as seen in FIG. 1, medical device 100 may include rotation actuator 112. In an exemplary configuration, handle body 110 may have a recess, cavity, or opening that accepts or otherwise receives at least a portion of rotation actuator 112. Rotation actuator 112 may be located at or near a distal end of handle assembly 122 and may control the rotation of one or more components or portions of distal portion 120 of medical device 100 (e.g., end effector assembly 118). Rotation actuator 112, and its interaction with other components of medical device 100, and its use are described further below.

Still referring to FIG. 1, and as noted above, medical device 100 includes shaft 116. Shaft 116 extends from a distal end of handle assembly 122 and rotation actuator 112 to distal portion 120 of medical device 100. Shaft 116 may be an outer tube with a sufficient length to reach target sites within the body of a subject. Additionally, shaft 116 may be flexible enough to navigate through tortuous anatomical pathways in various body lumens. As shown in FIG. 1, rotation actuator 112 may be coupled to a proximal portion of shaft 116 via a strain relief 114. Strain relief 114 may extend from a distal end of handle assembly 122 and overlap with a proximal portion of shaft 116, which may help to maintain the coupling of handle assembly 122 (e.g., including rotation actuator 112) to shaft 116. Strain relief 114 may be integrated or connected to handle assembly 122 by adhesive(s), overmold, press-fit, or any other ways commonly used in the art.

A protrusion 115 may also extend from the distal end of rotation actuator 112 and radially surround strain relief 114. Protrusion 115 may be cylindrical. In some aspects, protrusion 115 may help to provide structural support for strain relief 114 and/or shaft 116. In addition, shaft 116 may extend through strain relief 114 and terminate within protrusion 115. A proximal portion of shaft 116 may be secured within strain relief 114 or protrusion 115, for example, via one or more of glue, cladding, press fitting, or any other methods commonly used in the art.

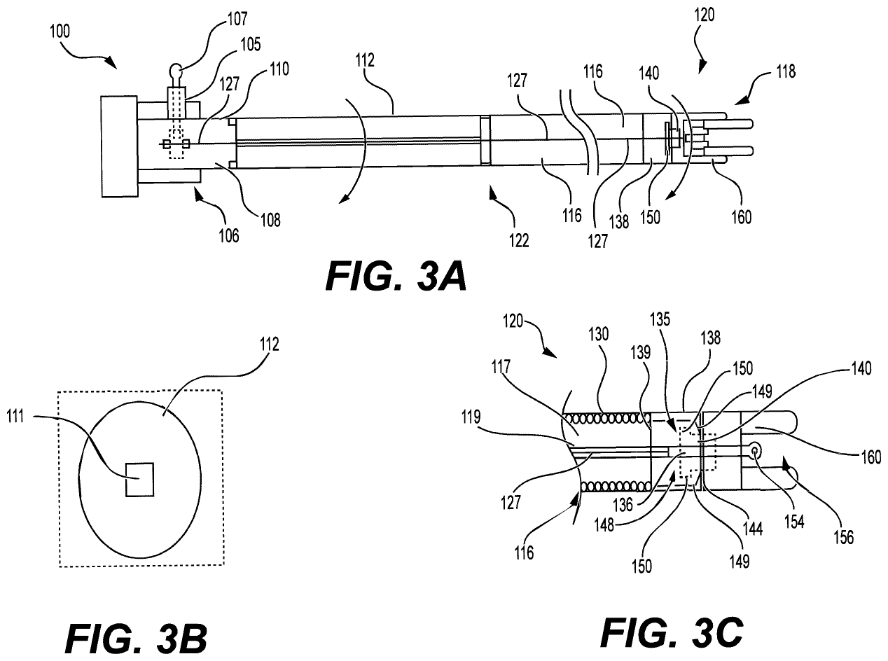

Wire 127 may be fixedly coupled to actuator 108 in various manners, including adhesives, a press-fit, ultrasonic welding, and laser welding, or any other commonly known methods in the art. Wire 127 may extend distally through an opening in strain relief 114 to a lumen 117 of shaft 116, such that wire 127 is moveable (e.g., longitudinally) relative to shaft 116 within lumen 117 (FIG. 3C). In other words, wire 127 may be translatable or slidable within lumen 117 of shaft 116. For example, the distal movement of actuator 108 results in the movement of wire 127 in a first direction, towards a distal end of handle assembly 122, and in a second direction, towards a proximal portion of handle assembly 122.

As noted above, actuator 108 may also include electrical insert 107 within cylindrical protrusion 105. FIG. 2 shows an exemplary cross-sectional view of a portion of handle assembly 122, showing components that are interior of handle body 110. Electrical insert 107 extends through protrusion 105 and into actuator 108. Electrical insert 107 may be coupled, for example via a screw connection, to an electrically conductive electrical connector 109. Electrical connector 109 includes a through-hole 111 that extends in a direction of the longitudinal axis of handle assembly 122. The proximal portion of wire 127 may be exposed from an insulative sheath at its proximal portion and may extend via through-hole 111. Additionally, electrical connector 109 and wire 127 may form a gap 125 and gap 125 may help to allow for rotation of wire 127 relative to electrical connector 109. During axial rotation of wire 127, electrical connector 109 and wire 127 may remain in electrical contact.

Furthermore, at least one or more cannula(s) 128 may be coupled (e.g., fixedly coupled) to the outer diameter or surface of wire 127. The one or more cannula(s) 128 may abut a distal end and/or a proximal end of the electrical connector 109, which may help to fix and prevent axial movement of wire 127 relative to the electrical connector 109. The proximal portion of the conductive wire 127 may extend into actuator 108 and may be fixedly coupled to electrical insert 107 by one or more extension(s) or radially widened portions (e.g., on proximal and distal sides the portion of wire 127 that is coupled to electrical insert 107). For example, at least one or more cannula(s) 128 may abut one or more extension(s) and/or may otherwise help to impart movement from actuator 108 to conductive wire 127. The electrical connector 109 may help to provide an electrical interface or connection between electrical insert 107 and the one or more cannulas 128 and wire 127 such that, when electrical insert 107 is energized or activated (receives electrical current from a connected current source), the one or more cannula(s) 128 and wire 127 are also energized or activated. The connection between these components may provide the electrical current to end effector assembly 118 of medical device 100.

In the embodiment, rotation actuator 112 may be coupled to handle body 110 in a manner permitting axial rotation of rotation actuator 112 relative to handle body 110 and shaft 116 but limiting axial movement of actuator 112 relative to handle body 110. For example, rotation actuator 112, may be coupled to handle assembly 122 as described for the distal components shown in FIG. 3B.

Moreover, when actuating rotation actuator 112, the connections between wire 127 and actuator 112 facilitate rotation of end effector assembly 118. For example, when rotation actuator 112 is rotated clockwise, wire 127 rotates clockwise, subsequently rotating aspects of end effector assembly 118 coupled to a distal end of wire 127 clockwise as well. Similarly, when rotation actuator 112 rotates counterclockwise, wire 127 rotates counterclockwise, and aspects of end effector assembly 118 coupled to a distal end of wire 127 rotate counterclockwise as well. As mentioned, wire 127 may have a sheath 119, which may be insulative. Sheath 119 may extend from a distal end of wire 127 to a proximal portion of wire 127. Sheath 119 may terminate at a location distal to actuator 108 to expose wire 127 and permit the connections of wire 127 to other components within handle assembly 122.

FIG. 3A shows a schematic view of medical device 100. As described below, distal components of distal portion 120 facilitate the rotation of end effector assembly 118. As noted, the proximal portion of rotation actuator 112 may be coupled to handle body 110 similarly to the distal components in FIG. 3B. For example, including a bushing and a flared cannula or any suitable connection allowing for the rotation of rotation actuator 112 and conductive wire 127, relative to handle body 110. As noted, conductive wire 127 may extend axially from a proximal end of rotation actuator 112 to a distal end of rotation actuator 112 and shaft 116 through which wire 127 extends. Within rotation actuator 112, through-hole 111 may be rectangular and receive the same or similarly shaped wire 127. As described below, wire 127 may be fixedly coupled to rotation actuator 112, such that rotation actuator 112 may transmit or otherwise impart rotation to wire 127.

FIG. 3B, illustrates a transverse or lateral cross-sectional view of rotation actuator 112. The transverse section of rotation actuator 112 may have a square, rectangular, triangular, or non-rounded circular shape. In the embodiment, rotation actuator 112 includes through-hole 111 with a square, rectangular, or triangular cross-section. Correspondingly, wire 127 may have a square, rectangular, or triangular shape such that the shape of wire 127 may be fixedly coupled to through-hole 111 within rotation actuator 112. Accordingly, when rotation actuator 112 rotates, wire 127 rotates. However, the disclosure is not so limited, and wire 127 and rotation actuator 112 may be any suitable corresponding shape. Alternatively, wire 127 may be rounded, while through-hole 111 within rotation actuator 112 may be square, rectangular, or triangular. In an embodiment, a square, rectangular, or triangular cannula corresponding to the shape of through-hole 111 of rotation actuator 112 may be welded or otherwise fixed to wire 127 such that the cannula facilitates the connection between wire 127 and rotation actuator 112.

FIG. 3C shows distal portion 120 of medical device 100, including aspects that may facilitate rotation of end effector assembly 118, for example, 360 degrees about its axis, relative to handle body 110 and shaft 116 of medical device 100. End effector assembly 118 may include various components, including tools, portions, parts, etc. to connect the tools to other parts of medical device 100 and permit the various functions of those tools. Exemplary tools include, but are not limited to, a tissue grasper (such as an electrocautery/coagulation grasper), a knife, biopsy forceps, scissors, a retrieval device (such as a net or a basket), electrocautery tools, etc. For example, end effector assembly 118 may include an electrocautery/coagulation grasper, so that a user may grasp tissue (particularly bleeding tissue, for example) and deliver energy for procedures where coagulation may be helpful to reduce bleeding. During such procedures and when otherwise necessary, medical device 100 may be coupled to an electrical source to provide monopolar or bipolar energy to end effector assembly 118 (e.g., an electrical coupled to the electrical on handle assembly 122).

As described earlier, actuator 108 and rotational actuator 112 control the actuation (e.g., open/close movement) and rotation of end effector assembly 118. For instance, the connection between the actuators and the distal components—such as wire 127—transmits the actions of actuator 108 and rotational actuator 112 to achieve the corresponding functions at the distal end of the end effector assembly 118. The specific mechanisms that enable these functionalities are discussed in further detail below.

In some aspects, wire 127 extends distally beyond the distal-most end of shaft 116 and attaches to aspects of end effector assembly 118. The distal end of shaft 116, including a coil 130, may be attached to a bushing 138 by adhesive, press-fit, ultrasonic welding, laser welding, or any other mechanisms commonly known in the art. In other words, coil 130 may not move longitudinally (e.g., distally, or proximally) relative to bushing 138 because the distal end of coil 130 is coupled to the proximal-facing surface of the proximal end 139 of bushing 138. In the configuration shown in FIG. 3C, the positioning of the distal face of coil 130 helps to prevent any longitudinal movement of coil 130 relative to bushing 138 and other components of the end effector assembly 118. Furthermore, as will be described below, wire 127 may be coupled to bushing 138 and a cannula 140, for example, at least with a distal actuator 136.

Additionally, bushing 138 may include a cavity 148, for example, surrounded by a wall 149. Cavity 148 may be a partially concentric opening configured to receive a cannula 140. Bushing 138 may have an increased thickness in its proximal portion and may taper (i.e., decrease in thickness) in a distal direction, such that cannula 140 may be at least partially secured within cavity 148. For example, wall 149 may help to inhibit distal movement of cannula 140. Cavity 148 may also have a size and shape configured to help retain portions of cannula 140. Cannula 140 may be generally cylindrical and may include features that protrude or otherwise extend radially outward into the inner surface of cavity 148. Specifically, cannula 140 may have a flange 150 that extends radially outward. When cannula 140 is positioned within cavity 148, flange 150 may extend toward a gap 135 in cavity 148.

In some aspects, gap 135 may be a variable size. Gap 135 may be a space positioned between an outer edge of flange 150 and an inner surface of a wall 149 of cavity 148 and flange 150. Both of flange 150 and gap 135 are positioned on both sides of the proximal portion of cannula 140. Cannula 140 may be inserted into cavity 148 (e.g., from a proximal side of cavity 148), and cannula 140 may be secured to the proximal portion of bushing 138. For example, cannula 140 may be secured to bushing 138 through a press-fit or friction-fit connection, resulting in flange 150 being surrounded by wall 149 within cavity 148, but still allowing for the formation of gap 135. As will be explained in more detail below, flange 150 may move within gap 135 to transmit rotation to end effector assembly 118 and also may help to prevent the detachment of shaft 116 (including coil 130) from end effector assembly 118.

Distal portion 120 may also include a clevis 160. Cannula 140 may be fixedly coupled to the proximal portion of clevis 160. Cannula 140 may be fixedly coupled to clevis 160 by an adhesive, ultrasonic welding, laser welding, or other mechanisms generally known in the art. In some embodiments, bushing 138 and clevis 160 may be coupled to each other such that a separation 144 or disjunction is formed between a portion of bushing 138 and clevis 160. As described below, wire 127 and clevis 160 may move relative to bushing 138. Separation 144 may help isolate the movement transmitted to clevis 160 by cannula 140 and prevent bushing 138 and coil 130 from moving when wire 127 is actuated.

Wire 127 may extend through a portion of the bushing 138 and be fixedly coupled to distal actuator 136 in various ways, including through adhesives, a press-fit, ultrasonic welding, laser welding, or other commonly known methods and mechanisms in the art. Distal actuator 136 may extend through both bushing 138 and cannula 140, which may include an annular gap (e.g., space to allow for rotation) on either side of distal actuator 136 and both bushing 138 and cannula 140. Distal actuator 136 may extend proximally to bushing 138. Additionally, distal actuator 136 may extend distally at least until a distal end of distal actuator 136 is within a portion of space 156 formed by portions of clevis 160. Distal actuator 136 may include a pin slot 154, for example, at the distal end of distal actuator 136. Within space 156, a user may attach a medical tool or end effector to distal actuator 136 by introducing a pin into pin slot 154, allowing distal actuator 136 to transmit movement to the medical tool. In use, axial translation of wire 127 distally (e.g., toward distal portion 120) may cause distal actuator 136 to open the jaws of (or otherwise actuate) the medical tool. Conversely, the axial translation of wire 127 proximally (e.g., away from distal portion 120) may close the jaws of (or otherwise de-actuate) the medical tool.

As described above, end effector assembly 118 and the proximal portions of wire 127 may be rotated by rotation actuator 112, and the distal end of wire 127 is fixedly coupled to the proximal end of distal actuator 136. Therefore, when a user actuates rotation actuator 112, wire 127 and distal actuator 136 also rotate and transmit rotation to one or more distal components, for example, to cannula 140, clevis 160, and an attached medical tool. As a result of rotation of rotation actuator 112, wire 127, distal actuator 136, cannula 140, clevis 160, and the attached medical tool rotate relative to shaft 116, coil 130, and bushing 138. Cannula 140, which includes flange 150 extending radially outward from its proximal end, may help to facilitate this relative rotation, while also helping to maintain the coupling between the components of end effector assembly 118 (e.g., clevis 160) and those of shaft 116.

In other words, cannula 140 and flange 150 help end effector assembly 118 remain coupled to shaft 116, even under rotational or axial forces. For example, if a force causes wire 127 to be pulled distally, flange 150 of cannula 140 will move within cavity 148 until flange 150 abuts a portion of bushing 138 inside cavity 148, acting as a stop surface to help prevent further proximal movement. In this manner, cannula 140 serves as a stop surface by engaging the inner surface of bushing 138, helping to prevent unwanted movement.

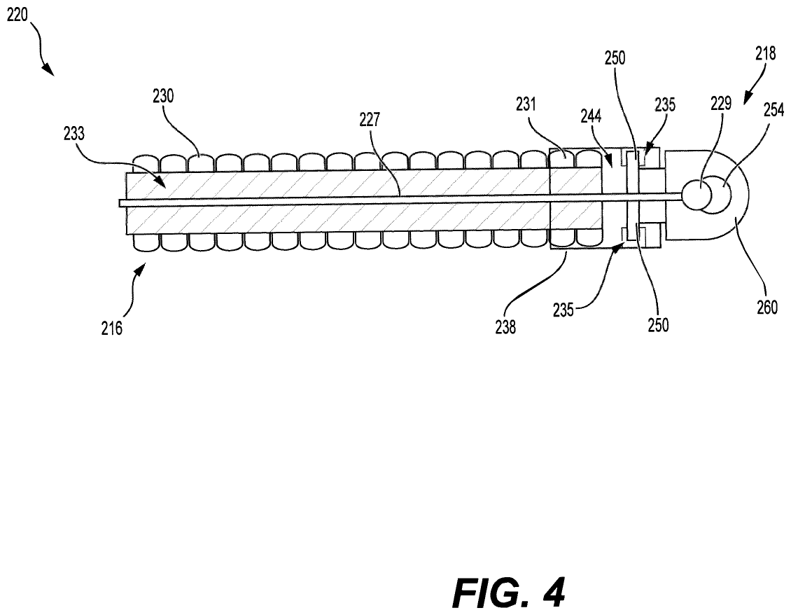

Aspects of the disclosure include methods of using device 100. To do so, an end effector may be coupled to the distal end of distal actuator 136. Subsequently, the user may introduce the distal portion 120 of medical device 100 into a GI tract via a natural orifice. The orifice can be, for example, the nose, mouth, or anus, and the placement can be in any portion of the GI tract, including the esophagus, stomach, duodenum, large intestine, or small intestine. Delivery and placement also can be in other body lumens or organs reachable via the GI tract, any other natural opening or body tract, bodily incision, or through a delivery device, such as an endoscope or sheath. Once the desired site is accessed, the user can actuate one or more actuators, including actuators 108 and 112, to control the actuation and/or the rotation of the distal end effector assembly 118 relative to the shaft 116 of the medical device 100. The embodiment of FIG. 4 and FIGS. 5A-5C may also use the exemplary method described above, except as shown and described below.

FIG. 4 depicts a cross-sectional view of a distal portion 220 of a medical device, in accordance with examples of this disclosure. As illustrated in FIG. 4, distal portion 220 is similar to distal portion 120. For example, distal portion 220 may be actuated (e.g., rotated in a similar manner) and may be electrically connected to a wire 227 and a handle assembly, similar to handle assembly 122, except as described herein. The handle assembly is not shown in FIG. 4 but may include, for example, a rotation actuator and an electrical insert positioned on another actuator (e.g., actuator 108). However, in this embodiment, the components of distal portion 220 may be modified and connected differently (e.g., to electrically isolate shaft 216) while maintaining the rotational functionality described above.

Similar to the distal end of shaft 116, shaft 216 includes a coil 230, and shaft 216 may radially surround a wire 227. Distal portion 220 may also include a bushing 238 and a clevis 260. However, unlike distal portion 120, distal portion 220 may not include cannula 140. For example, clevis 260 may include features similar to cannula 140.

In this embodiment, bushing 238 may be similar to bushing 138, except bushing 238 does not include cavity 148. Instead, the distal portion of bushing 238 may include a gap 235, which may accommodate portions of clevis 260. Clevis 260 may also be similar to clevis 160, except clevis 260 includes features that protrude radially outward into the gap 235 of bushing 238. Specifically, clevis 260 may include a flange 250 positioned at its proximal end.

Gap 235 may be a generally circular indentation or opening within a radially inner surface of bushing 238, and gap 235 may provide a space or a groove that may be used to couple clevis 260 to the distal portion of bushing 238. More specifically, flange 250 of clevis 260 extends radially outward from clevis 260 and within gap 235. As explained in more detail below, flange 250 may move (e.g., rotate) within gap 235 and may help to prevent the detachment of shaft 216 (including coil 230) from the end effector assembly 218 while maintaining relative rotation between the distal components (e.g., bushing 238).

In the embodiment, wire 227 may be similar to wire 127, except as described herein. Wire 227 extends from a handle (not shown) and distally beyond the distal-most end of shaft 216 and attaches to end effector assembly 218. Wire 227 may be coupled to clevis 260 or a distal actuator (e.g., similar to wire 127) in various ways, including through adhesives, a press-fit, ultrasonic welding, laser welding, or other commonly known methods and mechanisms in the art. In some embodiments, wire 227 may include a rounded end 229 configured to couple to a medical tool or end effector. Alternatively, rounded end 229 may help to couple wire 227 to a slot 254 on clevis 260. The distal portion of coil 230 may be coupled to bushing 238 by adhesive, press-fit, or any other way commonly known in the art. For example, bushing 238 may include internal threading that helps bushing 238 be screwed onto a distal portion 231 of coil 230. Alternatively, bushing 238 may be coupled to coil 230 using non-conductive glue. In the embodiment, distal portion 231 of coil 230 may be coupled to the proximal portion of bushing 238, such that coil 230 may not move distally. In other words, bushing 238 helps to prevent distal movement of coil 230 relative to other components of end effector assembly 218 (e.g., clevis 260).

As shown in FIG. 4, bushing 238 is fixedly coupled to coil 230, and bushing 238 is coupled to clevis 260, such that a separation 244 is formed distal to coil 230. For example, distal portion 231 of coil 230 or distal end of coil 230 may be adjacent to separation 244. Similar to separation 144, separation 244 helps isolate the movement transmitted to clevis 260 by wire 227, which may help prevent bushing 238 and coil 230 from moving when wire 227 is actuated. In addition, separation 244 may help isolate or insulate coil 230 and shaft 216 from electrical currents or other energy transmitted by wire 227 and clevis 260.

Additional features may be incorporated to enhance the electrical insulation of coil 230 and prevent induced currents from being transmitted from wire 227. For example, a non-conductive coating may be applied to or otherwise positioned around coil 230 to help the inner walls of coil 230 act as an insulated barrier against induced currents. The coating may be made of a polymeric film, for example, forming a thin layer along the inner circumference of coil 230. The coating may be applied or otherwise positioned around the entirety of coil 230, helping to mitigate unwanted electrical conduction. As shown in FIG. 4, an inner sheath 233 may also be positioned between wire 227 and coil 230. In some aspects, inner sheath 233 may be positioned along the inner circumference of coil 230. Inner sheath 233 may help to prevent currents that might otherwise be induced in coil 230 as a result of current being delivered through wire 227.

End effector assembly 218 may be rotated similarly to end effector assembly 118. For example, the proximal portions of wire 227 may be rotated by a rotation actuator (e.g., similar to rotation actuator 112). In the embodiment, when a user actuates a rotation actuator, wire 227, which may be coupled to clevis 260, transmits rotation to a medical tool coupled to clevis 260. As a result, wire 227, clevis 260 (i.e., including flange 250), and the attached medical tool rotate relative to shaft 216, coil 230, and bushing 238. Clevis 260, which includes flange 250 extending radially outward from its proximal end, facilitates this relative rotation while maintaining its coupling to bushing 238.

In other words, clevis 260 and flange 250 help end effector assembly 218 remain coupled to shaft 216 and coil 230, even under rotational forces. For example, gap 235 may help to provide a space for flange 250 to rotate but still provide a snug fit (e.g., longitudinally retain) for flange 250 of clevis 260 to remain coupled within gap 235. In some aspects, clevis 260 and flange 250 help end effector assembly 218 remain coupled to shaft 216 and coil 230 even under axial forces. For example, in some aspects, if a force causes wire 227 to be pulled proximally, flange 250 of clevis 260 may move slightly within gap 235 (within bushing 238) until flange 250 abuts a distal-facing portion of bushing 238, acting as a stop to prevent further proximal movement. In this manner, clevis 260 serves as a stop by engaging the distally-facing inner surface of bushing 238 within gap 235, preventing unwanted movement. Furthermore, during axial rotation of wire 227, an electrical insert similar to electrical insert 307 may remain in electrical contact with wire 227. For example, when the electrical insert is energized, wire 227 may also be energized. Wire 227 may transmit the energy delivered through wire 227 to an end effector, for example, to treat a tissue or treatment site.

FIGS. 5A-5C are cross-sectional views of various portions of a medical device, in accordance with examples of this disclosure. As illustrated in FIG. 5C, a distal portion 320 is similar to distal portion 120 and may be actuated (e.g., rotated in a similar manner), except as described herein. For example, distal portion 322 includes different mechanisms and features to control the rotation and actuation of, for example, a conductive wire 327 and a distal end effector assembly 318. Moreover, the handle components of the devices may be similar, for example, including a handle body 310, except as described further herein.

FIG. 5A shows an actuator 308 similar to actuator 108. Handle body 310 may include actuator 308 in the form of a spool, which may include an indented portion with a relatively smaller lateral cross-section, and extended portions with a relatively larger lateral cross-section. As discussed above, the indented portion may receive a user's fingers, allowing the user to control the movement of actuator 308. Similar to actuator 108, actuator 308 may be movable along a track (e.g., similar to track 113), allowing for longitudinal movement either distally or proximally. For example, the movement of actuator 308 may result in the actuation or de-actuation of end effector assembly 318 via conductive wire 327 (i.e., similar to conductive wire 127), as described above.

Actuator 304 may include an electrical insert 307 that is similar to electrical insert 107, as described above. For example, electrical insert 307 may be within a protrusion 305 that extends radially from a surface of actuator 304. Electrical insert 307 and protrusion 205 may be located at or near the proximal end of actuator 304. The proximal portion of the conductive wire 327 may extend into actuator 304 and be fixedly coupled to electrical insert 307 by one or more extension(s) 306 or radially widened portions (e.g., on proximal and distal sides the portion of wire 327 that is coupled to electrical insert 307). For example, at least one or more cannula(s) 328 (e.g., similar to cannula(s) 128) may abut one or more extension(s) 306. Alternatively, wire 327 may be fixed with a crimp, glue, or other manners known in the art. Conductive wire 327 may then extend distally to a distal end of distal portion 320, as described below.

Similar to the distal end of shaft 116, shaft 316 includes a coil 330, and shaft 316 may radially surround a wire 327. Distal portion 320 may also include a clevis 360. However, distal portion 320 may not include, for example, cannula 140 or bushing 238. Instead, distal portion 320 includes different mechanisms and features to insulate coil 330 from wire 327, and to control the rotation and actuation of end effector assembly 318.

The configuration and connections between conductive wire 327, shaft 316 (including coil 330), actuator 308, and handle body 310 help enable the rotation of conductive wire 327 when a knob 312 rotates. For example, when a user rotates knob 312 clockwise, shaft 316 and wire 327 rotate accordingly. In other words, the rotation of knob 312 also rotates wire 327 because wire 327 is fixedly coupled to a portion of shaft 316 (including coil 330), and shaft 316 is fixedly coupled to knob 312. As discussed in further detail below, the above components may rotate relative to handle body 310.

FIG. 5B is a cross-sectional view of the distal end of handle body 310 and an exemplary knob 312, which may be similar to rotation actuator 112, except as described herein. Knob 312 may be formed of two halves that couple to one another in any suitable way known in the art. The components of knob 312 and the distal components of handle body 310 facilitate the rotation of shaft 316 (including coil 330). As described below, when knob 312 is actuated, shaft 316 transmits rotation to wire 327, clevis 360, and a medical tool about its axis, relative to handle body 310 (including actuator 308), similar to handle body 110.

Handle body 310 may include an extension 311 that extends distally from a proximal or main portion of handle body 310. A portion of extension 311 may include protrusion(s) 350. Protrusion(s) 350 may extend radially outward relative to an outer surface of extension 311 to engage a portion of knob 312, as discussed below. Knob 312 may also define a recess 313 at its distal end, sized to accommodate a crimp 329. Crimp 329, or any similarly shaped cross-section, may be coupled to and/or surround shaft 316. Crimp 329 may correspond to the size or shape of recess 313 to sit in recess 313 and helps fixedly couple shaft 316 to knob 312. Because shaft 316 is coupled to knob 312, knob 312 will transmit rotation to other components such as wire 327, clevis 360, and a medical tool coupled to clevis 360.

Aspects of knob 312 will now be discussed in more detail. Knob 312 may include a proximal portion 339 and a nose portion 309. Nose portion 309 may be integral to proximal portion 339 of knob 312 and tapers to a narrow point at its most distal end. Knob 312 may also include a cavity 349 configured to accommodate extension 311 of handle body 310 with a matching size and shape. Additionally, knob 312 may have one or more opening(s) opposite the position of each protrusion(s) 350. In other words, knob 312 and extension 311 may be coupled by inserting protrusion(s) 350 into opening(s) within knob 312, such that extension 311 of handle body 310 may be nested within cavity 349 within knob 312.

Still referring to FIG. 5B, protrusion(s) 350 of extension 311 may extend radially outward into the opening(s) of knob 312. Once within the opening(s), protrusion(s) 350 may help to inhibit the removal of extension 311 of handle body 310 from knob 312. Although protrusion(s) 350 are not fixedly coupled to knob 312 (i.e., proximal portion 339), protrusion(s) 350 may help to longitudinally secure handle body 310 and extension 311 relative to knob 312. Protrusion(s) 350 help to prevent knob 312 from moving distally relative to handle body 310, while a small space surrounding the protrusion(s) 350 within the opening(s) of knob 312 may help to allow the rotation of knob 312 around protrusion(s) 350.

As shown in FIG. 5B, knob 312 may include a shaft opening 345 extending longitudinally through nose portion 309. Shaft opening 345 receives shaft 316 and wire 327 therein. Additionally, the handle body 310, including extension 311, includes a wire channel 315. Wire channel 315 extends distally to the distalmost end of handle body 310 (i.e., a distalmost end of extension 311) and longitudinally at a center of extension 311, to receive wire 327.

The position of shaft 316, coil 330, and conductive wire 327 within both extension 311 and knob 312 will be described in more detail. As seen in FIG. 5C, shaft 316 may be radially surrounded by a coil 330, which may be braided to help in torque transmission. Coil 330 may also include a coating similar to the non-conductive coating applied to coil 230. Shaft 316 may extend proximally through shaft opening 345 of knob 312 and the proximal portion of shaft 316 may extend into and sit within recess 313 of knob 312. In the embodiment, crimp 329 may be coupled to a radially outward-facing surface of shaft 316, which may help shaft 316 remain secured within recess 313. The radial width of crimp 329 may be equal to or slightly less than the radius of recess 313, forming a stable seating area that may help to prevent longitudinal movement of crimp 329 and shaft 316 relative to knob 312.

For example, when a distal or proximal force is applied to wire 327, crimp 329 may abut an inner surface of recess 313 in nose portion 309, helping to prevent any further distal or proximal movement of crimp 329 and shaft 316. Coil 330 of shaft 316 may be attached to clevis 360 by adhesive, press-fit, ultrasonic welding, laser welding, or any other method commonly known in the art. In other words, the distal face of coil 330 is coupled to the proximal-facing surface of the proximal portion 361 of clevis 360, helping the distal face of coil 330 to not move distally.

Wire 327 may be introduced into extension 311 and extend distally past both knob 312 and extension 311, continuing toward the distal end of distal portion 320, similar to wire 127 and wire 227 in previous embodiments. Shaft 316 and a sheath 319 similar to sheath 119, may also radially surround wire 327. As wire 327 extends distally beyond the distal-most end of shaft 316 and coil 330, wire 327 may be coupled to components of end effector assembly 318, such as clevis 360.

As shown in FIG. 5C, distal portion 320 may also include clevis 360, which may be similar to clevis 160 and clevis 260, except clevis 360 may include a proximal portion 361 with a rectangular cross-section and a distal portion 362 tapering in a distal direction and including a pin slot 354 similar to pin slot 254. Wire 327 extends to distal portion 362 of clevis 360, where wire 327 may be fixedly coupled to clevis 360. A user may attach a medical tool to clevis 360 by introducing a pin into pin slot 354, allowing wire 327 to transmit movement or energy to the medical tool, similar to the mechanism used with wire 127.

The combination and connections of these components allow for the rotation of knob 312, shaft 316, coil 330, and wire 327 relative to handle body 310. As previously described, protrusion(s) 350 of extension 311 engage with the opening(s) in knob 312, preventing axial movement while still facilitating rotation. Since protrusion(s) 350 are not fixedly attached to opening(s) in knob 312, knob 312 may rotate around the protrusion(s) 350 of extension 311. Additionally, crimp 329, positioned on shaft 316, secures shaft 316 within recess 313 of knob 312. When the user rotates knob 312, this rotational force transfers to shaft 316 and coil 330, which radially surround wire 327. Because wire 327 is coupled to clevis 360, clevis 360 (including a medical tool) will also rotate in unison with knob 312. Furthermore, during axial rotation of wire 327, electrical insert 307 may remain in electrical contact with wire 327. For example, when the electrical insert 307 is energized, wire 327 may also be energized. Wire 327 may transmit the energy delivered through wire 327 to an end effector, for example, to treat a tissue or treatment site, while being maneuverable.

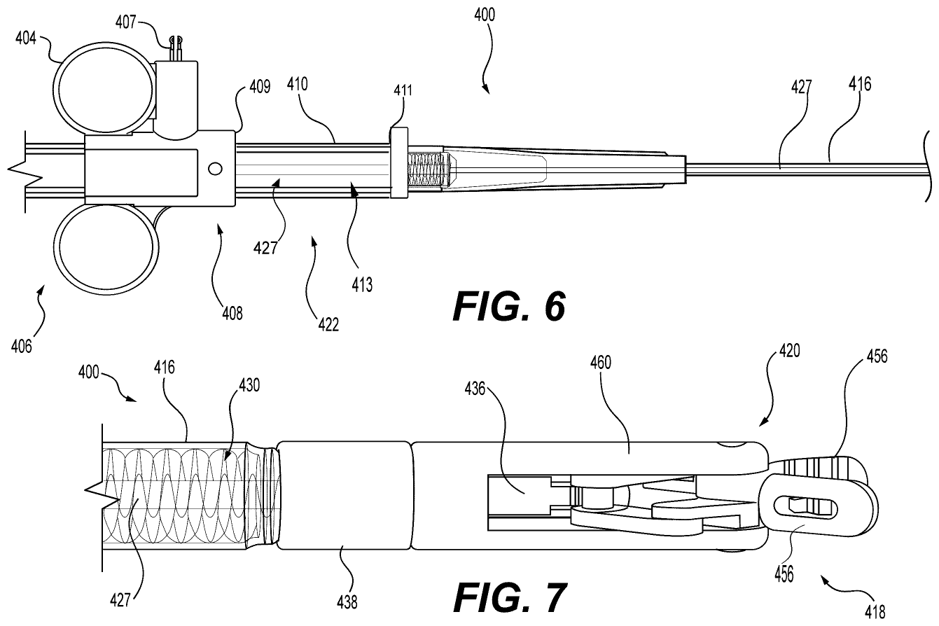

FIG. 6 and FIG. 7 are partially transparent views of portions of a medical device 400, in accordance with examples of this disclosure. As shown in FIG. 6 and FIG. 7, medical device 400 is similar to the medical devices described above, except as described herein. For example, medical device 400 includes a proximal portion 406 and a distal portion 420. Proximal portion 406 includes a handle assembly 422, which includes a handle body 410. However, unlike prior embodiments, medical device 400 does not include a rotation actuator (e.g., rotation actuator 112, FIG. 1). Instead, medical device 400 may use different mechanisms to control the rotation of components of distal portion 420, for example, such as a wire 427 and an end effector assembly 418.

Handle body 410 includes a longitudinal actuator 408, for example, similar to actuator 108. Longitudinal actuator 408 may include one or more finger rings or slot(s) 404 to help a user move actuator 408. Actuator 408 may be movable relative to handle body 410. For example, handle body 410 may include a track 413, and actuator 408 may be movable along track 413, allowing for longitudinal movement either distally or proximally. For example, the movement of actuator 408 may impart movement to wire 427, which may result in the actuation or de-actuation of end effector assembly 418 via wire 427 (e.g., similar to wire 127), as described above in relation to actuator 108. Actuator 408 may also include an electrical insert 407, similar to electrical insert 107. The proximal portion of wire 427 may extend into or otherwise be coupled to actuator 408 and be electrically coupled to electrical insert 407. Handle body 410 may include a shaft opening that receives proximal portions of shaft 416 and wire 427 therein. Wire 427 may extend distally from electrical insert 407 to a distal end of medical device 400, as described below.

The configuration and connections between wire 427, electrical insert 407, actuator 408, and handle body 410 help enable the rotation of wire 427 when handle body 410 rotates. For example, when a user rotates handle body 410 clockwise, wire 427, electrical insert 407, and actuator 408 rotate clockwise in unison. In other words, rotation of handle body 410 causes wire 427 to rotate because wire 427 is fixedly coupled to electrical insert 407 (e.g., also coupled to actuator 408), which is coupled to handle body 410. Actuator 408 may also include a surface 409 that may abut a corresponding surface 411 of handle body 410. Surface 409 and corresponding surface 411 may be flat surfaces (e.g., formed by portions of a square or rectangular cross-section), such that rotation of handle body 410 also rotates actuator 408. As discussed in more detail below, aspects of distal portion 420 may also rotate when a user rotates handle body 410. However, aspects or portions of distal portion 420 are not directly coupled to shaft 416 and handle body 410.

FIG. 7 is a partially transparent view of distal portion 420 of medical device 400, which is similar to distal portion 120, except as described herein. For example, distal portion 420 may include end effector assembly 418 (e.g., similar to end effector assembly 118). Distal portion 420 also includes a bushing 438, which may help to couple end effector assembly 418 to shaft 416 and handle body 410.

Shaft 416 may be similar to shaft 116, including a coil 430, which is coupled to a proximal end or portion of bushing 438 using adhesive, press-fitting, ultrasonic welding, laser welding, or any other suitable mechanism known in the art. Distal portion 420, including end effector assembly 418, may also feature a clevis 460 which may be similar to clevis 160. Clevis 460 may be located distal to and coupled to bushing 438 by similar mechanisms. As shown in FIG. 7, shaft 416 and clevis 460 are positioned on opposite sides of bushing 438, with shaft 416 connected to the proximal end of bushing 438 and with clevis 460 connected to the distal end of bushing 438. In other words, shaft 416 is indirectly coupled to clevis 460 through bushing 438, forming a separation between shaft 416 and end effector assembly 418. As discussed below, this separation may help isolate the proximal components (e.g., handle body 410, shaft 416, coil 430) from the components of distal portion 420 (e.g., end effector assembly 418).

As discussed above with respect to FIG. 3, clevis 460 (e.g., clevis 160) and bushing 438 (e.g., bushing 138) are rotatable relative to each other. Stated another way, clevis 460 and bushing 438 may be fixed longitudinally relative to one another but may be rotatably decoupled or uncoupled relative to one another. In these aspects, clevis 460 and bushing 438 may rotate freely relative to one another, such that end effector assembly 418 may rotate freely relative to shaft 416 and coil 430. However, in this embodiment, clevis 460 and bushing 438 rotate in the same direction in response to handle body 410. When a user rotates handle body 410, shaft 416 rotates, causing bushing 438 to rotate as well. At the same time, clevis 460 rotates due to the rotation of wire 427 and actuator 436, rather than directly from handle body 410. In other words, when the user rotates handle body 410 clockwise, shaft 416, bushing 438, wire 427, actuator 436, and clevis 460 all rotate clockwise (i.e., in the same direction) in response to the rotation of handle body 410.

Wire 427 may extend through bushing 438 and be fixedly coupled to distal actuator 436, similar to distal actuator 136. Distal actuator 436 is coupled to one or more links 456 (e.g., two links), such that links 456 pivot relative to distal actuator 436. Links 456 are connected to a pair of jaws 457 via axles, such that each jaw 457 pivots with its respective link. For example, axial translation of wire 427 moves actuator 436, causing one link 456 to move upward and the other downward, resulting in jaws 457 opening. Conversely, axial translation of wire 427 in the opposite direction (e.g., proximally) may close jaws 457. Alternatively, movement of wire 427 may otherwise actuate or control end effector assembly 418.

As described above, a user may manually rotate handle body 410. Wire 427 may be fixedly coupled to actuator 408, which in turn may be coupled to handle body 410. A proximal end or portion of shaft 416 may be coupled to handle body 410, and a distal end or portion of shaft 416 may be coupled to bushing 438. End effector assembly 418 (e.g., clevis 460) may be coupled to the distal end of bushing 438, positioned opposite shaft 416, and indirectly coupled to shaft 416. The combination and connections of these components helps the components—shaft 416, coil 430, wire 427, distal actuator 436, and bushing 438—rotate in response to handle body 410.

This configuration may facilitate the manual transmission of rotational force from handle assembly 422 to wire 427 and end effector assembly 418 without a rotation actuator. Additionally, since end effector assembly 418 may be indirectly coupled to shaft 416, strain on coil 430 may be reduced. For example, isolating end effector assembly 418 from coil 430 may help minimize the build-up of torque or tension that could otherwise accumulate in coil 430.

Accordingly, various aspects discussed herein may help improve a procedure's efficacy in treating a treatment site. Various aspects discussed herein may help to reduce or minimize the duration of the procedure and may help to reduce the risks of accidental manipulation by the user. Moreover, various aspects discussed herein may help to reduce the risk of induced currents to aspects of the medical device from a conductive wire. Furthermore, aspects discussed herein may help to reduce risks of accidental contact with tissue or other material during delivery, repositioning, or usage of a medical device in the procedure.

While principles of this disclosure are described herein with reference to illustrative aspects for various applications, it should be understood that the disclosure is not limited thereto. Those having ordinary skill in the art and access to the teachings provided herein will recognize additional modifications, applications, aspects, and substitution of equivalents all fall within the scope of the aspects described herein. Accordingly, the disclosure is not to be considered as limited by the foregoing description.

What is claimed is:

1. A medical device, comprising:
a shaft;
a wire, wherein the wire extends radially within the shaft;
an actuator;
an end effector assembly at a distal end of the shaft, wherein the wire is coupled to the actuator and the end effector assembly, wherein the wire transmits a force to the end effector assembly; and
a connection between the end effector assembly and the shaft, permitting axial rotation of the end effector assembly, the connection including:
a flange protruding radially outward relative to a position of the wire toward a gap; and
a clevis, wherein the wire is coupled to the clevis.

2. The medical device of claim 1, further comprising a bushing, wherein the shaft is coupled to the bushing, and wherein the bushing is distal to the shaft.

3. The medical device of claim 2, wherein the gap is defined between the flange and an inner surface of the bushing.

4. The medical device of claim 3, the flange being axially translatable relative to the shaft to engage a distally-facing inner surface of the bushing.

5. The medical device of claim 4, wherein the clevis and the wire rotate relative to the bushing and the shaft.

6. The medical device of claim 5, further comprising a cannula, wherein the flange protrudes from an inner surface of the cannula.

7. The medical device of claim 6, wherein the bushing includes a cavity, wherein the cannula is coupled to a radially outward facing surface of the cavity, and wherein the cannula is axially translatable relative to the shaft to engage a distally-facing inner surface of the cavity.

8. The medical device of claim 7, wherein the gap is defined between the flange of the cannula and the inner surface of the cavity of the bushing.

9. The medical device of claim 8, wherein the cannula is fixedly coupled to the clevis, and wherein a space separates the clevis and the bushing.

10. The medical device of claim 9, wherein the end effector assembly rotates when moved by the actuator, and wherein the clevis, the wire, and the cannula rotate relative to the shaft during rotation.

11. The medical device of claim 2, wherein the shaft includes a coil, wherein at least an inner surface of the coil includes a non-conductive coating, and wherein the bushing is fixed to an outer surface of a distal portion of the coil.

12. The medical device of claim 11, wherein the distal end of the clevis includes the flange, wherein the bushing includes the gap and the flange, wherein the flange extends radially outward from the clevis and within the gap, and wherein the distal portion of the coil is adjacent a space that separates the coil from the clevis.

13. The medical device of claim 1, wherein the medical device includes a handle body, and wherein the actuator is rotatably coupled to the handle body,
wherein the shaft is fixedly coupled to the actuator and extends to the end effector assembly for actuation of the end effector assembly, and
wherein the handle body includes an extension having a radial protrusion, wherein the actuator includes an opening that receives the radial protrusion.

14. The medical device of claim 13, wherein a crimp is positioned on the shaft and is positioned within a recess in the actuator.

15. The medical device of claim 14, wherein the actuator, the shaft, the wire, and the clevis rotate relative to the handle body and the extension of the handle body.

16. A medical device, comprising:
a handle body, wherein the handle body includes a rotation actuator and a longitudinal actuator, wherein the rotation actuator is rotatably coupled to the handle body, and wherein the longitudinal actuator is movably coupled to the handle body;
a wire, wherein a proximal portion of the wire is fixedly coupled to the longitudinal actuator, and wherein another portion of the wire is fixedly coupled to the rotation actuator,
a shaft, wherein the wire extends radially within the shaft; and
an end effector assembly, wherein the wire extends to the end effector assembly, the end effector assembly further comprising:
a bushing coupled to a distal end of the shaft, wherein the bushing includes a cavity;
a cannula positioned within the cavity of the bushing, wherein the cannula includes a flange extending radially outward toward a gap in the cavity; and
a clevis, wherein the wire is fixedly coupled to the clevis;
wherein movement of the longitudinal actuator moves the wire and the clevis longitudinally relative to the handle body, and wherein rotation of the rotation actuator rotates the wire, the cannula, and the clevis relative to the handle body, the shaft, and the bushing.

17. The medical device of claim 16, wherein a proximal portion of the bushing includes an increased thickness, wherein the bushing tapers in a distal direction, and wherein an inner wall of the bushing forms a stop surface configured to inhibit distal movement of the cannula.

18. The medical device of claim 17, wherein the gap is positioned between the flange and an inner surface of the wall of the cavity, wherein the cannula is fixedly coupled to a proximal portion of the clevis, wherein rotation of the rotation actuator rotates the wire and the clevis, and wherein rotation of the wire rotates the flange within the gap.

19. A medical device, comprising:
a handle body, wherein the handle body includes a longitudinal actuator;
a wire, wherein a proximal portion of the wire is fixedly coupled to the longitudinal actuator;
a shaft, wherein the wire extends radially within the shaft; and
an end effector assembly, wherein the wire extends to the end effector assembly, the end effector assembly further comprising:
a clevis, wherein the wire is fixedly coupled to the clevis;
a bushing coupled to a distal end of the shaft;
wherein the clevis is coupled a distal end of the bushing;
wherein rotation of the handle body rotates the shaft to rotate the bushing, and wherein rotation of the handle body rotates the wire to rotate the clevis.

20. The medical device of claim 19, wherein the shaft, the bushing, the wire, and the clevis rotate in a same direction.
